(19) United States
(12) Patent Application Publication
(10) Pub. No.: US 2015/0011646 A1
(43) Pub. Date: Jan. 8, 2015

(54) USAGE OF GUTTIFERONE K, A NATURAL COMPOUND FROM GARCINIA YUNNANENSIS HU ON TREATING HIGH METASTATIC ESOPHAGEAL CANCER

(71) Applicant: Hong Kong Baptist University, Hong Kong (HK)

(72) Inventors: Hongxi XU, Hong Kong (HK); Yuanzhi LAO, Hong Kong (HK); Zhichao XI, Hong Kong (HK); Hongsheng TAN, Hong Kong (HK); Kaixian CHEN, Hong Kong (HK); Zhaoxiang BIAN, Hong Kong (HK); Dajian YANG, Hong Kong (HK); Shilin CHEN, Hong Kong (HK); Aiping LU, Hong Kong (HK); Albert Sun Chi CHAN, Hong Kong (HK)

(73) Assignee: Hong Kong Baptist University, Hong Kong (HK)

(21) Appl. No.: 14/149,821

(22) Filed: Jan. 8, 2014

Related U.S. Application Data
(60) Provisional application No. 61/750,969, filed on Jan. 10, 2013.

(51) Int. Cl. A61K 31/122; A61K 36/38

(52) U.S. Cl. CPC A61K 31/122; A61K 36/38
USPC 514/681; 568/327

(57) ABSTRACT

This invention is related to compounds isolated from various Garcinia species and method of treating cancer. The present invention provides composition for treating cancer comprising polyprenylated acylphloroglucinol (PPAP) compound. The present invention also provides a composition comprising Guttiferone K for treating esophageal cancer.

### CROSS-REFERENCE TO RELATED APPLICATIONS

This application claims the benefit of U.S. Provisional Patent Application Ser. No. 61/750,969 filed Jan. 10, 2013, disclosure of which is incorporated herein by reference in its entirety.

### FIELD OF INVENTION

This invention relates to a chemical entity isolated from natural sources for its therapeutic uses. More particularly, it relates to a compound that is naturally occurring in the plant of Garcinia yunnanensis Hu and its biological activity of anticancer effects.

### BACKGROUND OF INVENTION

Esophageal cancer is one of the most common malignancies and is associated with a dismal prognosis. Although treatment options have increased for some patients, overall progress has been modest. Thus, there is a great need to develop new treatments. The present invention provides a natural compound, Guttiferone K, from Garcinia yunnanensis Hu and method of using the same for treating esophageal cancer by inhibiting the migration and invasion of esophageal cancer.

In the last decade, most of the research on Garcinia species has focused on the anticancer activity of gambogic acid (GA), a caged xanthone found at high concentrations in gamboge. GA has been involved in the injectable anticancer drug since the 1970s. In 2004, GA has been granted permission for testing in clinical trial as a wide spectrum anticancer drug. Gambogic acid and its derivatives are cytotoxic in many cancer cell lines by binding to the transferrin receptor and induction of G₂/M cell cycle arrest and mitochondrial and death receptor-mediated apoptosis. Gambogic acid also reduces invasion and angiogenesis, telomerase mRNA expression and activity and tumor volume in vivo. However, the anticancer effect of gambogic acid is not selective and it induces toxicity to the liver and kidney, which limits its development into a clinically useful anticancer drug.

Due to the toxicity of gambogic acid, there is a need for a novel and more selective compounds isolated from various Garcinia species for cancer treatment. This is an objective of this invention.

Citation or identification of any reference in this section or any other section of this application shall not be construed as an admission that such reference is available as prior art for the present application.

### SUMMARY OF INVENTION

Accordingly, the objective of this invention is to provide a compound from a natural source that exhibits potent anticancer effect and method of using the same for use in cancer treatment.

In accordance with one aspect of the present invention, there is provided a natural compound Guttiferone K, from Garcinia yunnanensis Hu, and method of using the same in treating esophageal cancer by inhibiting migration and invasion of esophageal cancer.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of Guttiferone K.

FIG. 2 shows the wound healing assay of Guttiferone K in ECA109 cells.

FIG. 3 shows the transwell assay of Guttiferone K in ECA109 cells. FIG. 3A is the statistical assay of migration cells in different Guttiferone K concentration treatment. FIG. 3B is the giemsa staining of migration cells in different Guttiferone K concentration treatment.

FIG. 4 shows the matrigel assay of Guttiferone K in ECA109 cells. FIG. 4A is the statistical assay of invasion cells in different Guttiferone K concentration treatment. FIG. 4B is the giemsa staining of invasion cells in different Guttiferone K concentration treatment.

### DETAILED DESCRIPTION OF INVENTION

The present invention is not to be limited in scope by any of the specific embodiments described herein. The following embodiments are presented for exemplification only.

Garcinia species (Family Guttiferae) are tropical evergreen trees and shrubs that are widely distributed in Southeastern Asia and their phytochemistry has been widely studied. Classic and caged xanthones have been isolated from various parts of these plants, and identified as their major bioactive components. Traditionally, Garcinia extract (called gamboge) has been used in folk and Chinese medicine to promote detoxification and treat inflammation and wounds, and xanthones isolated from various Garcinia species also showed antibacterial, antioxidant, antiviral and neuroprotective effects in Reutrakul, V.; Anantachoke, N.; Pohmakotr, M.; Jaipetch, T.; Sophasan, S.; Yoosook, C.; Kasisit, J.; Napaswat, C.; Santisuk, T.; Tuchinda, P., Cytotoxic and anti-HIV-1 caged xanthones from the resin and fruits of Garcinia hanburyi. Planta Med 2007, 73 (1), 33-40; and (a) Rukachaisirikul, V.; Phainuphong, P.; Sukpondma, Y.; Phongpaichit, S.; Taylor, W. C., Antibacterial caged-tetraprenylated xanthones from the stem bark of Garcinia scortechinii. Planta Med 2005, 71 (2), 165-70; (b) Jang, S. W.; Okada, M.; Sayeed, I.; Xiao, G.; Stein, D.; Jin, P.; Ye, K., Gambogic amide, a selective agonist for TrkA receptor that possesses robust neurotrophic activity, prevents neuronal cell death. Proc Natl Acad Sci USA 2007, 104 (41), 16329-34; (c) Sampath, P. D.; Kannan, V., Mitigation of mitochondrial dysfunction and regulation of eNOS expression during experimental myocardial necrosis by alpha-mangostin, a xanthonic derivative from Garcinia mangostana. Drug Chem Toxicol 2009, 32 (4), 344-52.

It is found by the inventors of the subject application that several polyprenylated acylphloroglucinol (PPAP) compounds had potent cytotoxic effects on human colorectal cancer cell lines without affecting the normal human colon fibroblasts. On human colon cancer HT-29 cell line, Guttiferone K is the most potent PPAP compound. The present invention provides a compound, Guttiferone K for use as an anticancer drug against esophageal cancer by significantly inhibiting esophageal cancer cell migration and invasion.

Extraction and Isolation.

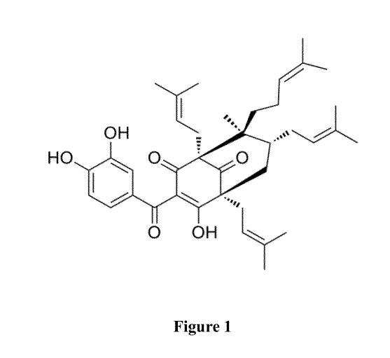

The air-dried and powdered pericarp (9.0 kg) of Garcinia yunnanensis Hu was extracted with acetone (20 L) at room temperature for three times. The extracted solution was evaporated under reduced pressure to yield a dark green residue (1.2 kg). The residue was chromatographed on silica gel eluted by CHC1₃, EtOAc, and acetone sequentially. The CHC1₃ fraction was evaporated in vacuum to give a residue (750 gram), part of which (400 gram) was subjected to silica gel column eluted with a gradient hexane/acetone system (100:0 to 0:100, v/v). Four fractions (I-V) were obtained on the basis of TLC analysis. Fraction II was separated using silica gel and eluted with gradient petroleum ether/acetone system (10:0 to 0:10) to produce four subfractions. The second subfraction was separated by preparative HPLC on an Alltima C-18 column eluted with CH₃CN in 0.1 acetic acid (0.1% acetic acid/CH3CN, 5/95) to yield Guttiferone K (3.0 g). The structure of Guttiferone K is shown in FIG. 1.

Cell Culture

Human esophageal cancer cell line ECA109 were maintained in RPMI1640 (Invitrogen) supplemented with 10% fetal bovine serum (Invitrogen), 100 U/ml penicillin and 100 μg/ml streptomycin, within a humidified atmosphere containing 5% CO2 at 37° C.

Anti-Migration Activity

Wound Healing Assay

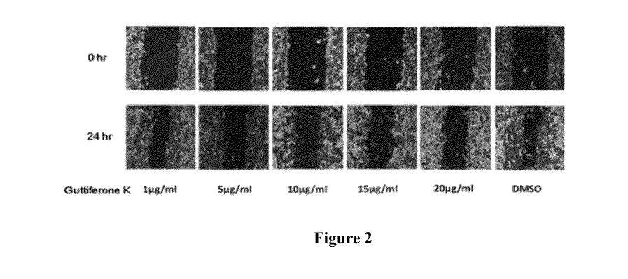

ECA109 cells were seeded on 12-well plates at a density of 1×10⁵ cells/well. After the cells reached sub-confluence, the mono-layer cells were wounded by scraping off the cells and then grown in medium for 24 hours. The migrated distance of cells was monitored and imaged under a microscope. As shown in FIG. 2, Guttiferone K suppresses the wound healing in a concentration dependent manner.

Transwell Assay

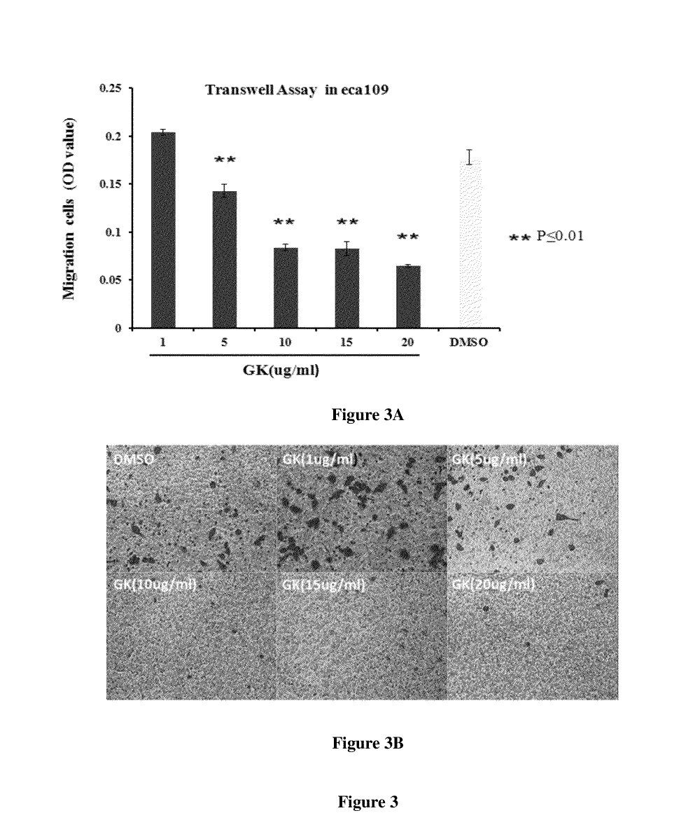

Cell migration was also determined using a transwell (Corning) with a pore size of 8 μm. 5×10⁴ cells were seeded in serum-free medium in the upper chamber, while medium containing 10% FBS in the lower chamber. After incubating for 24 hours at 37° C., cells in the upper chamber were carefully removed with a cotton swab and the cells that had traversed to reverse face of the membrane were fixed in methanol, stained with Giemsa, and counted. FIG. 3 shows the Guttiferone K suppresses ECA109 cells migration through transwell at concentration dependent manner.

Invasion Assay (Matrigel Assay)

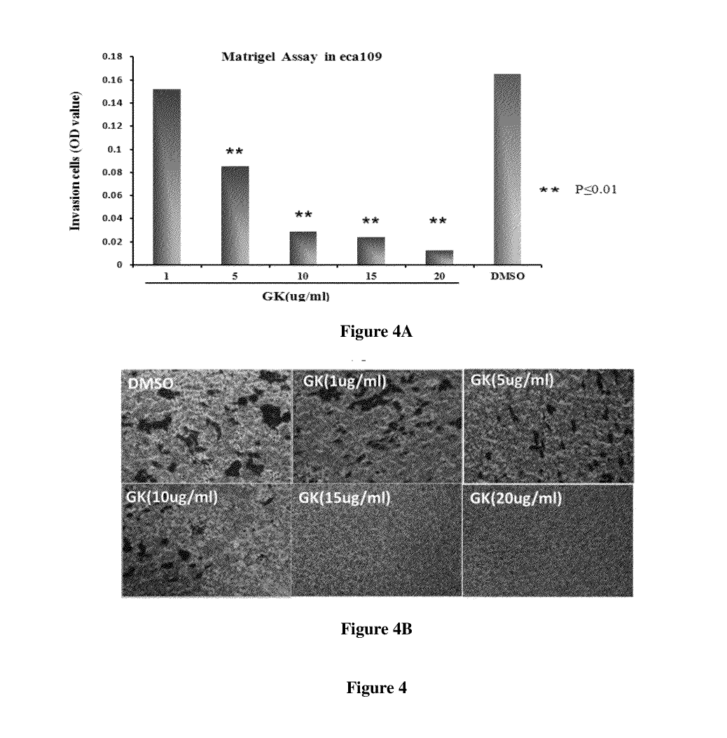

Cell invasion was determined using Matrigel (BD) coated transwell (Corning) with a pore size of 8 μm. 5×10⁴ cells were seeded in serum-free medium in the upper chamber, while medium containing 10% FBS in the lower chamber. After incubating for 72 hours at 37° C., cells in the upper chamber were carefully removed with a cotton swab and the cells that had traversed to reverse face of the membrane were fixed in methanol, stained with Giemsa, and counted. FIG. 4 shows the Guttiferone K suppresses ECA109 cells invasion through matrigel at a concentration dependent manner.

Plant Material

The pericarp of Garcinia yunnanensis Hu were collected in Luxi of Dehong prefecture, Yunnan province, China in 2006. The plant material was identified by Dr. Chunfeng Qiao. A herbarium sample was deposited in the Shanghai University of Traditional Chinese Medicine.

Discussion

In this invention, a natural compound, Guttiferone K, isolated from Garcinia yunnanensis Hu with anti-migration and anti-invasion effects on cancer cells is provided. Kan, W. L.; Yin, C.; Xu, H. X.; Xu, G; To, K. K.; Cho, C. H.; Rudd, J. A.; Lin, G, Antitumor effects of novel compound, guttiferone K, on colon cancer by p21Waf1/Cip1-mediated G(0)/G(1) cell cycle arrest and apoptosis. Int J Cancer 2012, shows that Guttiferone K has low toxicity in 10 mg/Kg concentration in mice. In summary, the present invention provides an anti-esophageal cancer composition comprises Guttiferone K and method of using the same for treating esophageal cancer.

### INDUSTRIAL APPLICABILITY

The present invention discloses a chemical entity isolated from natural sources for its therapeutic uses. More particularly, it relates to compound that is naturally occurring in the plant of Garcinia yunnanensis Hu and its biological activity of anticancer effects.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A composition for treating cancer comprising a compound with the chemical structure of

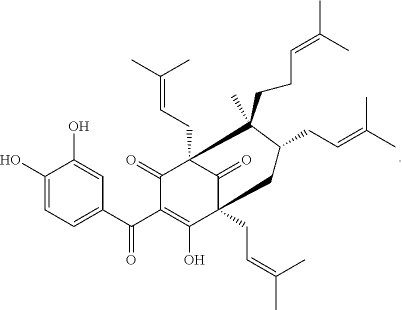 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]

2. The composition according to claim 1 wherein said compound comprising Guttiferone K.

3. The composition according to claim 1 wherein said compound is isolated from a natural plant.

4. The composition according to claim 3 wherein said natural plant comprising Garcinia species.

5. The composition according to claim 4 wherein said Garcinia species comprising Garcinia yunnanesis Hu.

6. The composition according to claim 1 wherein the treated cancer comprising esophageal cancers.

7. The composition according to claim 1 wherein the cancer treatment comprising anti-migration of cancer cells and anti-invasion of cancer cells.

8. A use of the composition according to claim 1 for manufacture of a medicament for cancer treatment.

9. The use according to claim 8 wherein said composition is used as a cancer metastasis inhibitor.

10. A method of treating cancer using composition according to claim 1 by administering said composition to a subject in need of such treatment.
